(19) United States
(12) Patent Application Publication
(10) Pub. No.: US 2025/0243325 A1
(43) Pub. Date: Jul. 31, 2025

(54) PHOSPHORUS-31 MRI AGENTS

(71) Applicant: UNIVERSITEIT TWENTE, Enschede (NL)

(72) Inventors: Olga KOSHKINA, Enschede (NL); Timo RHEINBERGER, Enschede (NL); Frederik WURM, Enschede (NL)

(21) Appl. No.: 18/858,949

(22) PCT Filed: May 8, 2023
(86) PCT No.: PCT/EP2023/062140

§ 371 Date: Oct. 22, 2024

(30) Foreign Application Priority Data
May 9, 2022 (NL) 2031805

(51) Int. Cl. C08G 79/04; A61K 49/12; A61K 49/18

(52) U.S. Cl. CPC C08G 79/04; A61K 49/12; A61K 49/1809

(57) ABSTRACT

Use of a P-containing polymer for measuring ³¹P-MRI, wherein the P-containing polymer is selected from polyphosphates, polyphosphonates, poly(phosphine oxide)s, polyphosphazenes, polyphosphinates, polyphosphoramidates, polyphosphorodiamidates, polyphosphoamides, polythionophosphates, and polythionophosphonates. A polyphosphonate copolymer and an aqueous suspension comprising micelles of the polyphosphonate copolymer.

### FIELD OF THE INVENTION

The present invention relates to use of a P-containing polymer for measuring ³¹P-MRI, to a polyphosphonate copolymer and an aqueous suspension comprising micelles of the polyphosphonate copolymer.

### BACKGROUND OF THE INVENTION

Magnetic Resonance Imaging (MRI) is an anatomical imaging technique, that produces an image based on the magnetic resonance properties of tissues. Different to other techniques, such as radioactive imaging (e.g. PET) and optical imaging, MRI is free of ionizing radiation and is not limited by tissue penetration depth. The contrast of the images may be increased by using contrast agents, which are often gadolinium-based. Gd(III) is a paramagnetic ion that can modulate the relaxation properties of water protons, making the spots with high Gd concentration appear brighter (so-called T₁-weighed imaging). Such contrast agents raised toxicological concerns, after reports of gadolinium accumulation in brain and tissues, and ecological concerns due to the increasing levels of gadolinium detected in rivers and oceans. Superparamagnetic iron oxide nanoparticles are alternative contrast agents, but these also showed organ accumulation; the FDA therefore retracted many iron oxide formulations.

Hot Spot MRI introduced the imaging of other nuclei, such as fluorine-19, which can act as a selective “color” label on an anatomical proton image. As the hot-spot nucleus is detected directly, the signal from hot-spot nucleus can be quantified from the signal-to-noise ratio of an MRI image. Hot spot MRI is for example effective in visualizing the processes behind cardiovascular diseases and inflammation, in labeling of immune cells, and in detection of tumors.¹⁻⁷ As the hot spot agents are free of metals, they could become a solution to the problems of proton MRI contrast agents.

However, the current hot-spot agents are also still far from ideal. ¹⁹F MRI agents typically contain liquid per- and polyfluoroalkyl substances (PFAS), which are typically formulated as a nano emulsion or nanoparticle. PFAS display an extraordinarily high chemical stability, and are not degraded in vivo. Moreover, they are both hydrophobic and lipophobic; this poor solubility can lead to a prolonged accumulation in the organs, up to several months. Such compounds are also considered major environmental pollutants. Furthermore, the presence of the PFAS-phase can make the co-encapsulation of additional cargo, such as drugs and other therapeutic molecules challenging. Particularly in emulsions, the additional cargo can be encapsulated only in the thin surfactant layer.

Phosphorus-31 is a natural, non-radioactive NMR active isotope of phosphorus with a nuclear spin of ½ and 100% natural abundance. Phosphorous-31 is omnipresent in biomolecules, for example nucleic acids. However, the sensitivity of phosphorus-31 is only about 8% of that of hydrogen-1. The low sensitivity and often very short relaxation times make the majority of biomolecules not detectable. Nevertheless, ³¹P MR spectroscopy can be used to monitor phosphorylation in muscles and to sense the pH of cells. As a result, MRI scanners equipped with a ³¹P coil are readily available. To date, however, the low sensitivity of the ³¹P-nucleus remained the major challenge in the development of exogenous ³¹P imaging agents, as imaging has a lower sensitivity than spectroscopy.

### SUMMARY OF THE INVENTION

It is an objective of the present invention to overcome one or more of the abovementioned drawbacks or at least to provide a useful alternative. It is a further objective of the present invention to provide an MRI hot spot agent which does not accumulate in the human body. It is a further objective of the present invention to provide an MRI hot spot agent which is biocompatible. It is a further objective of the present invention to provide an MRI hot spot agent which is biodegradable. It is a further objective of the present invention to provide an MRI hot spot agent with an increased signal-to-noise ratio as compared to ³¹P in biomolecules. It is a further objective of the present invention to provide an MRI hot spot agent which can be used to encapsulate drugs and/or other therapeutic molecules.

Thereto, the present invention provides for the use of a P-containing polymer for measuring ³¹P MRI, preferably as a hot-spot agent, wherein the P-containing polymer is selected from polyphosphates, polyphosphonates, poly(phosphine oxide)s, polyphosphazenes, polyphosphinates, polyphosphoramidates, polyphosphorodiamidates, polyphosphoamides, polythionophosphates, and polythionophosphonates. P-containing polymers provide for a high local P-concentration due to the localization of all ³¹P atoms in the polymers. Moreover, the polymers used according to the invention all contain phosphorus-31 in their polymer backbone, such that an especially high concentration of phosphorus-31 may be achieved. ³¹P MRI images may be acquired via direct imaging such as mCSSI (multi-echo chemical shift selective imaging) or RARE (Rapid Acquisition with Relaxation Enhancement), or via spectroscopic imaging such as CSI (Chemical Shift Imaging). Preferably the ³¹P MRI images are acquired via direct imaging.

Preferably, the amount of P in the polymer is at least 3 wt. %, more preferably at least 5 wt. %, even more preferably at least 10 wt. %, most preferably at least 15 wt. %.

Preferably, the P-containing polymer is comprised in a colloidal system (i.e. a state of subdivision, implying that molecules or polymolecular particles dispersed in a medium have at least in one direction a dimension roughly between 1 nm and 1 μm, or that in a system discontinuities are found at distances of that order; IUPAC, Compendium of Chemical Terminology, 2nd ed. (the “Gold Book”), Compiled by A. D. McNaught and A. Wilkinson. Blackwell Scientific Publications, Oxford (1997), Online version (2019-) created by S. J. Chalk. ISBN 0-9678550-9-8, https://doi.org/10.1351/goldbook), such as in a micelle dispersed in a liquid medium. Preferably the liquid medium is water or a physiological solution. Preferably, the P-containing polymer is comprised in a micelle which has a diameter of between 1 nm and 1 μm, more preferably between 2 nm and 500 nm. In case of a non-spherical micelle, the before mentioned diameter is the maximum diameter of the particle. This especially ensures a high concentration of ³¹P nuclei, further overcoming the sensitivity issues. Colloidal systems are easily injectable in the human body and often provide for better biocompatibility than systems with larger particles.

Different P-containing polymers may be used for forming such colloids. For example, the P-containing polymer may be insoluble in water. Such a hydrophobic P-containing polymer may be formed into an emulsion with the aid of an emulsifier. The P-containing polymer may also be water soluble, in which case the P-containing polymer may be formed into colloidal systems by cross-linking of the polymer chains. Cross-linking may either be chemical, or by physical interactions between the polymer chains, Amphiphilic copolymer assemblies contain at least two types of monomers. One or both monomers may be P-containing. The P-containing monomer may be relatively hydrophilic, and the other monomer may be relatively hydrophobic. If the relatively hydrophilic monomer is P-containing, the relatively hydrophobic monomer may be styrene. Alternatively, the P-containing monomer may be relatively hydrophobic, and the other monomer may be relatively hydrophilic. In such a case, the hydrophilic monomer may for example be poly(ethylene glycol) (PEG). Alternatively, the P-containing polymer may be a copolymer of at least two P-containing monomers, wherein one of the monomers is relatively hydrophilic as compared to the other P-containing monomer, which is relatively hydrophobic.

In aqueous solution, copolymers, such as block copolymers and gradient copolymers, may form core-shell micelles with a relatively hydrophobic core and a hydrophilic shell. Importantly, the sphere of hydrophilic polymers stabilizes the hydrophobic core and may stabilize other nanocarriers in physiological milieu, ensuring a so-called stealth effect.

Nanocarriers, such as micelles, comprising a hydrophobic polymer and/or amphiphilic co-polymer may additionally be used to encapsulate additional cargo that is not bio-available otherwise, such as hydrophobic drugs and other therapeutic agents.

Advantageously, the P-containing polymer is an amphiphilic polymer which forms core-shell micelles in an aqueous solution, such as a physiological solution, wherein the hydrophilic shell is relatively rich in hydrophilic P-containing monomeric units. The higher mobility of the polymer chains in the hydrophilic shell and hydrophobic core has proven to be advantageous for the signal-to-noise ratio of ³¹P MRI imaging.

Preferably, the P-containing polymer is biocompatible and/or biodegradable, wherein biodegradable means that the P-containing polymer is fully broken down into components which can be excreted by the human body within 1 year. More preferably, the P-containing polymer is both biocompatible and biodegradable.

Preferably, the P-containing polymer has a T_{g} below 37° C., such as below 35° C., preferably below 30° C., more preferably below 25° C. or below 20° C., wherein the T₃ is measured upon heating the P-containing polymer at 10° C./min, Such polymers are in a “liquid-like” viscous state when injected in the body, i.e. at body temperature, and thus during MRI measurement. This positively affects the longitudinal and transverse relaxation times T₁ and T₂, which together with concentration determine the signal intensity. Most preferably, the P-containing polymer has a T_{g} below 0° C.

In MR spectroscopy, following a pulse that flips the nuclear spin in a certain angle, typically 90°, the magnetization of an element precesses around the z axis and at the same time returns to its equilibrium along the z-axis. The transverse x- and y-components of M (the total magnetization of the spins) decay towards zero with a characteristic time T₂. The longitudinal z-component grows back to its starting position with time constant T₁.

Longitudinal relaxation times T₁ may be measured using an inverse recovery sequence (well-known to the skilled person, e.g. described in: NMR in Biological Systems: From Molecules to Human, K.V.R. Chary, Girjesh Govil, Springer, 2008). For the measurements of transverse relaxation times T₂, the Carr-Purcell-Meiboom-Gill (CPMG) sequence, which is a standard sequence preinstalled on NMR spectrometers (also described in before mentioned publication) may be used.

The signal-to-noise ratio (SNR) in hot-spot MRI depends on T₁ and T₂, which depend on the properties of molecule. A shorter T₁ and a longer T₂ typically lead to a higher imaging contrast. Thus, for an optimal signal-to-noise ratio, it is advantageous that T₁ is as short as possible, and that T₂ is as long as possible.

Preferably, T₁ is at most 5 s, such as at most 3 s or 2.5 s. Preferably, T₂ is at least 0.03 s, such as at least 0.04 s or 0.045 s.

Preferably, the P-containing polymer has a T₁ between 0.05 s and 5 s, a T₂ between 0.03 s and 3 s, and wherein T₁>T₂; more preferably a T₁ between 0.1 s and 3 s, and a T₂ between 0.04 s and 1.5 s, and wherein T₁>T₂; and more preferably a T₁ between 0.5 s and 2.5 s, and a T₂ between 0.045 and 1.5, and wherein T₁>T₂; wherein T₁ and T₂ are the longitudinal and transverse relaxation times calculated using a mono-exponential decay fit.

Preferably, the chemical shift of the ³¹P in the P-containing polymer is higher than 4 ppm, such as higher than 6 ppm or higher than 10 ppm, ensuring that the polymer is distinguishable from the ³¹P chemical shifts of endogenous background molecules such as ATP and DNA, which typically have a lower chemical shift. Preferably, the chemical shift of the P-containing polymer is between 4-100 ppm, even more preferably between 6-60 ppm, yet more preferably between 10-50 ppm, most preferably between 20 and 40 ppm.

Preferably, the P-containing polymer is a polyphosphonate (PPn). Polyphosphonates are biocompatible polymers from the class of polyphosphoesters. The chemical shift of PPns can be adjusted between 10-200 ppm, ensuring that the polymer is distinguishable from the ³¹P chemical shifts of endogenous background molecules such as ATP and DNA. For example, the ³¹P chemical shift in ethyl-PPn is 37 ppm and in phenyl-PPn it is 22 ppm.

Furthermore, polymers from the group of polyphosphoesters, such as the polyphosphonates presented in this invention, are known to be excellent carriers for other cargo, specific hydrophobic cargo, such as medicines and other therapeutic molecules.

Furthermore, polyphosphoesters, and thus polyphosphonates, are biodegradable. Moreover, the degradation speed can be tuned when needed by adjusting the chemical composition.

Preferably, the polyphosphonate is a copolymer. More preferably, the polyphosphonate is an amphiphilic copolymer. An amphiphilic copolymer comprises hydrophilic monomeric units and lipophilic/hydrophobic monomeric units. In aqueous solution, such polymers may form micelles with a hydrophobic core and a hydrophilic shell.

Preferably, the polyphosphonate copolymer comprises at least two monomeric units A and B, wherein monomeric unit

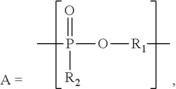

and monomeric unit

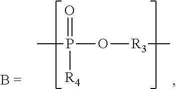

and
wherein R₁ and R₃ each independently are

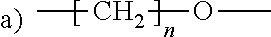

wherein n>1, preferably wherein 1<n<10; or CH₃

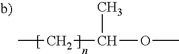

wherein n>0, preferably wherein 0<n<10; or

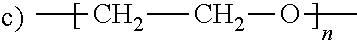

wherein n>0, preferably wherein 0<n<10; or CH₃

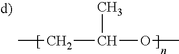

wherein n>0, preferably wherein 0<n<10;
   and wherein
   R₂ represents optionally substituted phenyl, optionally substituted benzyl, or optionally substituted phenethyl; and
   R₄ represents straight or branched C₁₋₆ alkyl, straight or branched C₂₋₆ alkenyl, straight or branched C₁₋₆ alkoxy, or straight or branched C₁₋₆ alkanoyl.

More preferably, R₁ and R₃ are a) with n=2;
R₂ represents methylphenyl, dimethylphenyl, ethylphenyl, methylbenzyl, or phenethyl; and
R₄ represents straight or branched C₁₋₄ alkyl, straight or branched C₂₋₄ alkenyl, straight or branched C₁₋₄ alkoxy, or straight or branched C₁₋₄ alkanoyl.

Most preferably, R₂ represents phenyl, and
R₄ represents methyl or ethyl, preferably ethyl.

Preferably, the polyphosphonate copolymer comprises from 30-70 mol % A, and from 30-70 mol % B, preferably from 35-65 mol % A, and from 35-65 mol % B, most preferably from 40-60 mol % A, and from 40-60 mol % B, the total of A and B adding up to 100 mol %.

Preferably, the polyphosphonate copolymer comprises between 10 and 1000 monomeric units, more preferably between 30 and 500 monomeric units, and even more preferably between 50 and 400 monomeric units.

Preferably, the polyphosphonate copolymer has a molecular weight (Mₙ) between 1000 and 100.000 g/mol, more preferably between 3000 and 80.000 g/mol, and even more preferably between 5000 and 60.000 g/mol. Preferably, the polyphosphonate copolymer has a dispersity D (Mw/M, between 1.01 and 10.00, more preferably between 1.02 and 8, and even more preferably between 1.03 and 6.

Preferably, the polyphosphonate copolymer is a diblock copolymer, a normal tapered block copolymer, or a gradient copolymer. In the case of copolymers with a hydrophilic monomer, such as monomeric unit B, and a hydrophobic monomer, such as monomeric unit A, such copolymers with the abovementioned compositions form core-shell micellar structures in aqueous solution. In a core-shell micellar structure, the core is relatively rich in the most hydrophobic monomer, in this case monomeric unit A, whereas the shell is relatively rich in the most hydrophilic monomer, in this case monomeric unit B.

Core-shell micelles have the advantage that hydrophobic cargo such as medicines and other therapeutic agents may be added to the core. Furthermore, especially the high mobility of hydrophilic units in the shell leads to a better imaging resolution.

It has proven to be especially advantageous if the copolymer is a gradient copolymer. In this case, the higher percentage of hydrophilic monomer units in the hydrophobic core is thought to lead to an increased mobility of polymer chains in the core as well, which positively influences the relaxation times T₁ and T₂, and thereby the imaging resolution of the monomer units in the core. Also, the monomer units in the shell have a higher mobility and therefore a better imaging signal.

Specifically, the present invention also relates to a polyphosphonate copolymer, which comprises two monomeric units A and B, wherein
monomeric uni

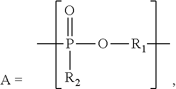

and
monomeric unit

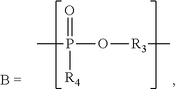

and wherein
R₁ and R₃ are

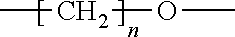

wherein n=2-4, and wherein
R₂ represents phenyl, benzyl, or phenethyl; and
R₄ represents methyl, ethyl or propyl,
wherein the copolymer comprises from 40-60 mol % A, and from 60-40 mol % B, and
wherein the polyphosphonate copolymer is a diblock copolymer, a normal tapered block copolymer, or a gradient copolymer, preferably wherein the polyphosphonate copolymer is a gradient copolymer.

More specifically, the present invention also relates to a polyphosphonate copolymer, which comprises two monomeric units A and B, wherein
monomeric unit

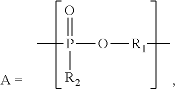

and
monomeric unit

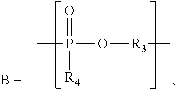

and wherein
R₁ and R₃ are

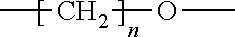

wherein n=2, and wherein
R₂ represents phenyl;
R₄ represents ethyl; and
wherein the copolymer comprises from 40-60 mol % A, and from 60-40 mol % B, and
wherein the polyphosphonate copolymer is a diblock copolymer, a normal tapered block copolymer, or a gradient copolymer, preferably wherein the polyphosphonate copolymer is a gradient copolymer.

Gleede et al, (Gleede, T.; Markwart, J. C.; Huber, N.; Rieger, E.; Wurm, F. R., in: Competitive Copolymerization: Access to Aziridine Copolymers with Adjustable Gradient Strengths. Macromolecules 2019, 52 (24). 9703-9714) define four groups of gradient structures via r-parameters (reactivity parameter), which is a common way to characterize copolymers. For statistically polymerized copolymers, the four groups are defined as:

| | |
|---|---|
| Soft gradient for | 0 < Δr ≤ 1.5 |
| Medium gradient for | 1.5 < Δr ≤ 7.5 |
| Hard gradient for | 7.5 < Δr ≤ 25 |
| Block(-like) for | 25 < Δr |

wherein Δr is the difference between the reactivity parameter of monomer A, r_{A} and the reactivity parameter of monomer B, r_{B}, wherein the reactivity parameters are calculated by taking the average value of at least three values calculated following the instructions of Jaacks,¹¹ Frey¹², BSL¹³ and/or the Meyer-Lowry¹⁴ model: the standard deviation has to be below 5%.

Preferably, the polyphosphonate copolymer is a gradient copolymer which has a gradient as defined in Gleede et al. of block(-like), medium or hard, preferably of medium or hard.

Preferably, the polyphosphonate copolymer is a gradient copolymer wherein r_{A} is between 3.5 and 25.0, and wherein r_{B} is between 0.02 and 0.5, more preferably wherein r_{A} is between 4.0 and 24.7, and wherein r_{B} is between 0.03 and 0.4, most preferably wherein r_{A} is between 4.3 and 24.4, and wherein r_{B} is between 0.03 and 0.25.

Shull, Interfacial activity of gradient copolymers, Macromolecules 2002, 35, 8631-8639 describes a gradient parameter λ for symmetric copolymers. Gradient parameter A describes the length of the composition gradient relative to the entire length of the copolymer. For AB copolymers, when λ=0, the copolymer is a conventional block copolymer consisting of separate blocks of A and B units. When λ=1, the composition varies smoothly along the chain from pure A to pure B.

Preferably, the polyphosphonate copolymer is a gradient copolymer which has a value A relative to the length of the minority monomeric unit as defined by Shull et al. of 0.1-1, more preferably 0.2-0.99, most preferably of 0.3-0.98.

The present invention also relates to an aqueous suspension, preferably in a physiological saline solution, comprising micelles of the polyphosphonate copolymer. Preferably, the micelles have a hydrodynamic radius Rₕ of between 5 and 100 nm, more preferably of between 6 and 90 nm, more preferably of between 7 and 80 nm, as measured by DLS according to ISO 22412:2008.

Preferably, the PDI of the micelles is between 0,001-0.5 as defined by ISO 22412:2008, more preferably between 0.01-0.5, even more preferably between 0.01-0.2.

Preferably, the concentration of the micelles in the aqueous suspension is at least 1 mg/mL, more preferably at least 5 mg/mL, most preferably at least 10 mg/mL. A higher concentration leads to an increased MRI visibility. It is particularly advantageous if the concentration of the micelles in the aqueous suspension is between 1-500 mg/mL, preferably 5-490 mg/mL, more preferably 10-480 mg/mL.

The present invention particularly relates to an aqueous suspension comprising micelles of the polyphosphonate copolymer, wherein the micelles have a Rₕ of between 5 and 100 nm as defined by ISO 22412:2008, a PDI of 0.001-0.5 as defined by ISO 22412:2008, and a concentration of between 1-500 mg/mL, preferably 5-490 mg/mL, more preferably 10-480 mg/mL.

### BRIEF DESCRIPTION OF THE FIGURES

FIG. 1 depicts the chemical structure and a schematic representation of phenyl-co-ethyl phosphonate gradient co-polymer micelles.

FIG. 2 depicts the results of a toxicity study of phenyl-co-ethyl phosphonate gradient copolymer.

FIG. 3 depicts ¹H and ³¹P MRI images of micelles of a block copolymer of PEG and PhPPn.

FIG. 4 depicts ¹H and ³¹P MRI images of micelles of a block copolymer of PS and PEtPPn.

FIG. 5 depicts ¹H and ³¹P MRI images of micelles of phenyl-co-ethyl phosphonate gradient copolymer.

FIG. 6 depicts the MRI results of injection of micelles according to the invention in the physalis.

### DETAILED DESCRIPTION OF THE INVENTION

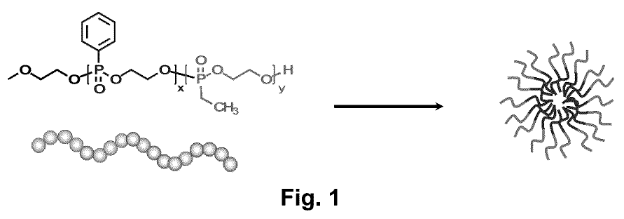

FIG. 1 depicts the chemical structure and a schematic representation of PhPPn-grad-EtPPn copolymer and its micelles.

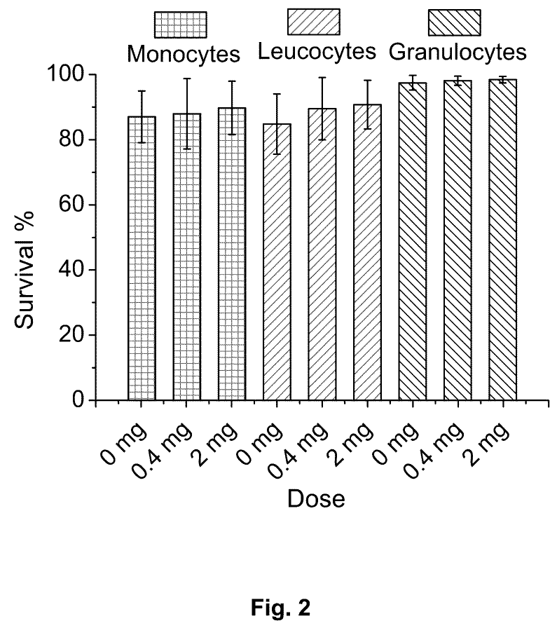

FIG. 2 shows the result of a cell viability study with different concentrations of PhPPn₃₀-grad-EtPPn₃₀ micelles and monocytes, leukocytes and granulocytes.

FIG. 3 depicts MRI images of a PEG₅₀₀₀-b-P(PhPPn)₃₀ micelle, with from left to right: an image based on ¹H as a reference, and an image based on the frequency of ³¹P. The concentration of micelles was 40 mg/mL, and the concentration of ³¹P was 3.5 mg/mL. The SNR was 7.9.

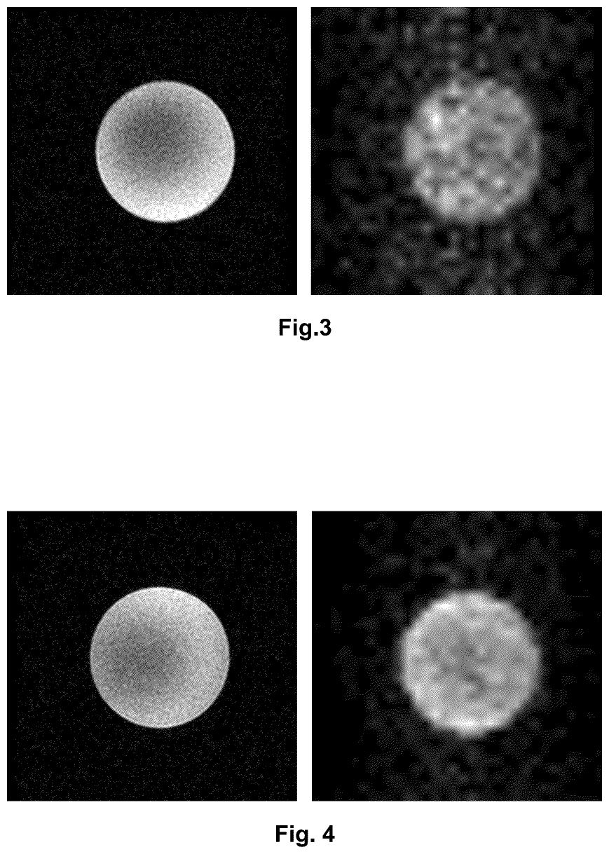

FIG. 4 depicts MRI images of a PEtPPn₆₂-b-PS₃₄₅ micelle, with from left to right: an image based on 1H as a reference, and an image based on the frequency of ³¹P. The concentration of micelles was 200 mg/mL, and the concentration of ³¹P was 11 mg/mL. The SNR was 18.3.

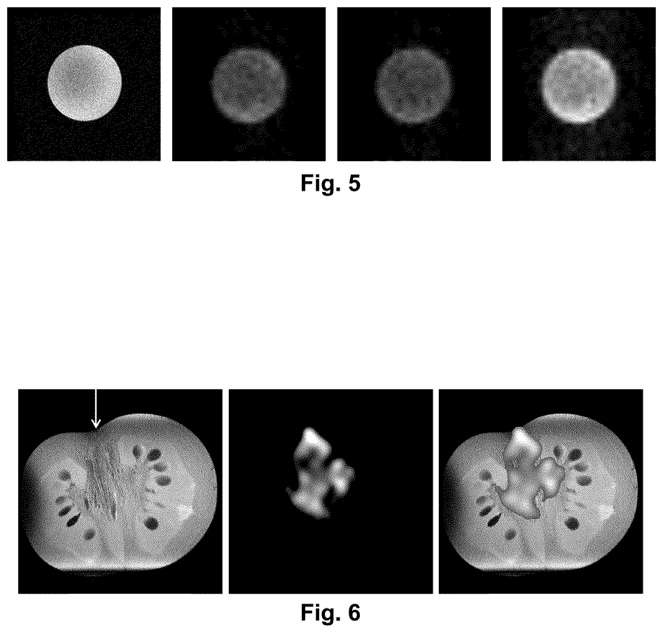

FIG. 5 depicts MRI images of a PhPPn₃₀-grad-EtPPn₃₀ micelle, with from left to right: an image based on ¹H as a reference, an image based on the frequency of ³¹P in EtPPn, an image based on the frequency of ³¹P in PhPPn, and the ³¹P sum image. The concentration of micelles was 38 mg/mL, and the concentration of ³¹P was 7.7 mg/mL. The SNR was 26.4.

FIG. 6 depicts MRI images of PhPPn₃₀-grad-EtPPn₃₀ micelles injected into a physalis, with from left to right: an image based on 1H as a reference, the ³¹P sum image, and a merged image showing the ³¹P sum image overlayed on the ¹H based image. The arrow denotes the site of injection of the micelles.

### EXAMPLES

### Materials

Solvents and chemicals were purchased from Acros Organics, Sigma Aldrich or Fluka and used as received unless otherwise stated. All chemicals were purchased in the highest purities, dry and stored over molecular sieves (4 Å), if possible. Ultrapure water with a resistivity of 18 MΩ cm⁻¹ (Milli-Q, Millipore®) was used for the self-assembly experiments. 2-(Benzyloxy)ethanol was acquired from ABCR, distilled from calcium hydride and stored over molecular sieves (3 and 4 Å) and under inert gas prior to use. DBU was purchased from Sigma Aldrich, distilled from calcium hydride and stored over molecular sieves (3 and 4 Å) and under inert gas prior to use. Deuterated solvents were purchased from Deutero GmbH (Kastellaun, Germany) or Merck and used as received.

### Methods

Size exclusion chromatography (SEC) measurements were performed in DMF (containing 1 g·L⁻¹ of LiBr) at 60° C. and a flow rate of 1 mL mirn⁻¹ with a PSS SECcurity as an integrated instrument, including three PSS GRAM column (100/1000/1000 g mol⁻¹) and a refractive index (RI) detector. Calibration was carried out using poly(ethylene glycol) or polystyrene standards supplied by Polymer Standards Service. The SEC data were plotted with OriginPro 9 software from OriginLab Corporation.

NMR spectroscopy was measured at Brucker Avance III 400 MHz spectrometer equipped with a PA BBO 400S1 BBF-H-D-05 Z SP probe at 298 K. As deuterated solvents CDCl₃, CD₂Cl₂ or D₂O were used. The proton spectra were calibrated against the solvent signal (CDCl₃: δ H=7.26 ppm, CD₂Cl₂: δ H=5.32 ppm, D₂O: δ H=4.79 ppm). Longitudinal relaxation times T₁ were measured using an inverse recovery sequence. For the measurements of transverse relaxation times T₂ the Carr-Purcell-Meiboom-Gill (CPMG) sequence, as included in the Bruker Topspin software, was used. At least 10 data points were acquired, which were then used for data fitting. An interscan delay was set to 5×T₁. In case of samples in water, deuterium oxide (10 vol %) was added to the samples for locking. Data analysis was performed using Mestrenova14 from Mestrelab.

Dynamic Light Scattering (DLS) to measure Rₕ was done at a Zetasizer Lab from Malvern UK at a scattering angle of 900, and 295 K. The samples were diluted with ultrapure water so that the attenuator was at the step 10-11 (set automatically by the device). Data analysis was done with ZSxplorer 2.2.0.147 software from Malvern Panalytical

Magnetic Resonance Imaging (M R) images were recorded at a vertical Bruker AVANCE^{III} and AVANCE NEO 9.4T wide bore NMR spectrometers driven by ParaVision 5.1 and 360 v3.0, respectively, and operating at frequencies of 400.2 MHz for ¹H and 162.0 MHz for ³¹P measurements. Experiments were carried out using a Bruker microimaging unit (Micro 2.5) equipped with actively shielded gradient sets (capable of 1.5 T/m maximum gradient strength and 150 μs rise time at 100% gradient switching) and a dual tunable ¹H/³¹P 25-mm birdcage resonator. Due to the vertical orientation of the MRI system, all phantoms were scanned in an upright position.

Anatomical reference images were acquired by standard FLASH or RARE (FLASH: FOV 20×20 mm², matrix 128×128, TE 1.62 ms, TR 60 ms, ST 2 mm, NA 1, TAcq 8 sec; RARE: FOV 30×30 mm², matrix 256×256, TE 4.39 ms, TR 4 sec, ST 0.75 mm, RARE factor 4, NA 1, TAcq 4 min 16 sec). Subsequently, polyphosphonates were detected by using 2D ³¹P chemical shift imaging (CSI) or multi-chemical selective imaging (mCSSI). The slice used for spectroscopic imaging (6-10 mm) was placed in axial orientation covering a large part of the phantom, or physalis. The 2D ³¹P CSI data sets were recorded with a sine-bell acquisition-weighted sequence to improve the spatial response function using the following parameters: flip angle, 45°; TR, 250 ms; matrix 32×32; data points in the spectral domain, 1024; spectral width, 6510 Hz: slice selection with a 500-μs sinc3 pulse; TAcq, 8 min. Data sets were analyzed by an in-house-developed software module¹⁷ based on the LabVIEW package (National Instruments, Austin). An exponential filter of 20 Hz was applied in the spectroscopic direction.

mCSSI was carried out as described previously¹⁸ using selective exciting frequencies with a bandwidth of 913 Hz (gauss 3 ms): For P(PhPPn₃₀-grad-EtPPn₃₀) 3030 163.0138883 and 162.0110640 MHz, PEG₅₀₀₀-b-P(PhPPn₃₀) 162.0107971 MHz, PEtPPn₆₂-b-PS₃₄₅ 162.0140139 MHz (TE 6.32 ms, TR 2.5 sec, RARE factor 32, matrix 32×32, ST 8 mm, effective spectral bandwidth 15000 Hz, NA 200, TAcq 8 min 20 sec.

Ceil viability—To obtain circulating immune cells, heparinized blood was withdrawn by venous puncture of the inferior vena cava of a mouse. Blood was collected via a 23G cannula in heparin-aerated collection tubes. Erythrocytes were lysed by adding the 4-fold amount of ammonium chloride buffer (pH 7.4). After 10 min of incubation at room temperature the samples were centrifuged at 350×g for 10 min at 20° C.

To determine cell toxicity of 3P micelles, 1×10⁶ murine immune cells were incubated with either 10 or 50 μL/mL in DMEM and incubated for 1 h at 37° C. Afterwards, cells were washed and stained with CD45, CD11 b and Ly6G (all 1:100) to discriminate monocytes (CD45⁺, CD11b⁺, Ly6G⁻), lymphocytes (CD45⁺, CD11 b⁺, Ly6G⁻) and granulocytes (CD45⁺, CD11b⁺, Ly6G⁺). To determine the number of dead cells, samples were taken up in MACS buffer with 1 μg/mL DAPL. Cells were gated with appropriated FSC/SSC settings and the number of DAPL⁻ cells were determined via flow cytometry.

Differential Scanning Calorimetry (DSC) measurements were performed using a Trios DSC 25 series thermal analysis system the temperature range from −80° C. to 500° C. under nitrogen with a heating rate of 10° C. min⁻¹. All glass transition temperatures (T_{g}) were obtained from the second heating ramp of the experiment.

### Monomer Synthesis

Ethyl phosphonic dichloride (EtPCI)⁸

A mixture of diethyl-ethylphosphonate (252.9 g, 1.521 mol) and DMF (1.3 mL) were added dropwise to refluxing thionyl chloride (305 mL, 4.2 mol). Strong gas evolution of ethylene chloride and sulfur dioxide indicated the progress of the reaction. After 16 hours the gas evolution declined. To complete the reaction the bath temperature was increased to 120° C. for 24 h. The thionyl chloride was separated via distillation. Two times of fractionated distillation of the raw product yielded the desired dichloride as a yellowish liquid (202.2 g, yield 100%, bp 40-42° C./7·10⁻² mbar).

¹H NMR (CDCl₃, ppm): δ=2.6 (dq, ²J_{HP}=15.0 Hz, ³J_{HH}=7.5 Hz, 2H, —P—CH₂—), 1.4 (dt, ³J_{HP}=30.1 Hz, ³J_{HH}=7.5 Hz, 3H,methyl group).

³¹P{H} NMR (CDCl₃, ppm): δ=53.7.

### 2-Ethyl-2-oxo-1,3,2-dioxaphospholane (1) (EtPPn)⁸

A flame-dried three-necked round-bottom flask, equipped with a magnetic stirring bar and two dropping funnels, was charged with 400 mL of dry THF and cooled to −21° C. Ethylphosphonic dichloride (153.4 g, 1.04 mol) was dissolved in dry THF (400 mL) and transferred into one dropping funnel via a flame-dried stainless steel capillary. A solution of dry ethylene glycol (64.8 g, 1.04 mol) and dry pyridine (165.1 g, 2.08 mol) in THF (300 mL) was transferred into the second dropping funnel via a flame-dried stainless steel capillary. A slow dropping speed was adjusted to be approximately equal for both mixtures. After complete addition the solution was stirred for 1 h and kept overnight at −80° C. to facilitate the precipitation of the pyridinium hydrochloride byproduct. The precipitate was removed by filtration via a flame-dried Schlenk funnel, and the solvent was removed at reduced pressure. Two times of fractionated distillation yielded the desired product as colorless oil (86.3 g, yield 61%, bp 61° C./2.1·10⁻³ mbar).

¹H NMR (CDCl₃, ppm): δ=4.6-4.0 (m, 4H, —CH₂—CH₂—), 1.9 (dq, ²J_{HP}=18.3 Hz, ³J_{HH}=7.8 Hz, 2H, —P—CH₂—), 1.1 (m, 3H, —CH₃).

³¹P{H} NMR (CDCl₃, ppm): δ=52.5.

### Methylphosphonic dichloride (MePCI)⁹

A mixture of dimethyl-methyl phosphonate (114.5 g, 0.92 mol) and DMF (0.9 mL) were added dropwise to refluxing thionyl chloride (160 mL, 2.2 mol). Strong gas evolution of methyl chloride and sulfur dioxide indicate the progress of the reaction. After 12 hours the gas evolution declined. To complete the reaction the bath temperature was increased to 120° C. for 24 h. The thionyl chloride was separated via distillation. Two times of fractionated distillation of the raw product yielded the desired dichloride as colorless crystals (34.7 g, yield: 28%, bp. 48-50° C./1·10⁻³ mbar).

¹H NMR (, ppm): δ=2.5 (d, ²J_{HP}=16.4 Hz, 3H, —CH₃).

³¹P{H} NMR (CDCl₃, ppm): δ=43.7

### 2-methyl-1,3,2-dioxaphospholane 2-oxide (4) (MePPn)⁹

A flame-dried three-necked round-bottom flask, equipped with a magnetic stirring bar and two dropping funnels, was charged with 100 mL of dry THF and cooled to −21° C. Methylphosphonic dichloride (34.7 g, 261 mmol) was dissolved in dry THF (250 mL) and transferred into one dropping funnel via a flame-dried stainless steel capillary. A solution of dry ethylene glycol (16.2 g, 261 mmol) and dry pyridine (41.3 g, 521 mmol) in THF (250 mL) was transferred into the second dropping funnel via a flame-dried stainless steel capillary. A slow dropping speed was adjusted to be approximately equal for both mixtures. After complete addition, the solution was stirred for 1 h and stored over night at −80° C. to facilitate the precipitation of the pyridinium hydrochloride byproduct. The precipitate was removed by filtration via a flame-dried Schlenk funnel and the solvent was removed at reduced pressure. Fractionated distillation yielded the desired product as colorless crystals (8.2 g, yield: 27%, b.p. 80′C/1·10⁻² mbar).

¹H NMR (CDCl₃, ppm): δ=4.5-4.1 (m, 4H, —CH₂—CH₂—), 1.6 (d, ³J_{HP}=17.6 Hz, 3H, —CH₃).

³¹P {H} NMR (CDCl₃, ppm): δ=48.7.

### 2-phenyl-1,3,2-dioxaphospholane 2-oxide (PhPPn)

PhPPn was performed according to a modified literature protocol⁹ A flame-dried three-necked round-bottom flask, equipped with a magnetic stirring bar and two dropping funnels, was charged with 100 mL of dry THF and cooled to −21° C. Phenylphosphonic dichloride (50.8 g, 260 mmol) was dissolved in dry THF (250 mL) and transferred into one dropping funnel via a flame-dried stainless steel capillary. A solution of dry ethylene glycol (16.2 g, 260 mmol) and dry pyridine (41.2 g, 521 mmol) in THF (250 mL) was transferred into the second dropping funnel via a flame-dried stainless steel capillary. A slow dropping speed was adjusted to be approximately equal for both mixtures. After complete addition, the solution was stirred for 1 h and stored over night at −80° C. to facilitate the precipitation of the pyridinium hydrochloride byproduct. The precipitate was removed by filtration via a flame-dried Schlenk funnel and the solvent was removed at reduced pressure. Fractionated distillation yielded the desired product as colorless solid (34.3 g, yield: 71%, b.p. 113-115° C./1·10⁻³ mbar).

¹H NMR (CDCl₃, ppm): δ=7.8 (dd, ⁴J_{HP}=14.2 Hz, ³J_{HH}=6.9 Hz, 2H, aromatic protons ortho), 7.6-7.4 (m, 3H, aromatic protons meta, para), 4.8-4.3 (m, 4H, —O—CH₂—CH₂—O—)

³¹P{H} NMR (CDCl₃, ppm): δ=36.0.

### Example 1: PPn Gradient Copolymer Micelles

### PPn Gradient Copolymer Synthesis

Ring-Opening Polymerization Catalyzed with DBU

Polymerization was performed according to a modified literature protocol.¹⁰ The particular monomers were weighed in a flame-dried Schlenk-tube, dissolved in dry benzene and dried by lyophilization. The monomer was dissolved in dry dichloromethane to a total concentration of 4 mol L⁻¹. A stock solution of initiator 2-methoxyethanole in dry dichloromethane was prepared with a concentration of 0.2 mol L⁻¹ and the calculated amount was added to the monomer solution. A stock solution of DBU in dry dichloromethane was prepared with a concentration of 0.2 mol L⁻¹. The monomer solution and the catalyst solution were set to the respective reaction temperature (in general −10° C.).

The polymerization was initiated by the addition of the calculated volume of catalyst solution containing 3.0 equivalents of DBU in respect to the initiator. Polymerization was terminated by the rapid addition of an excess of formic acid dissolved in dichloromethane with a concentration of 20 mg mL⁻¹. The colorless, amorphous polymers were purified by two times precipitation in cold diethyl ether and dried in vacuo. Yields ranged from 70% to 95%.

Representative NMR data of P(PhPPnₙ-grad-MePPnₘ):

¹H NMR (CDCl₃, ppm): δ=7.9-7.6 (m, aromatic protons ortho), 7.6-7.3 (m, aromatic protons meta, para), 4.4-3.9 (m, backbone —CH₂—), 3.3-3.2 (m, initiator —CH₃), 1.6-1.2 (m, P—CH₃)

¹³C {H} NMR(CDCl₃, ppm): δ=132.9 (s, broad, aromatic-C-para), 131.7 (s, broad, aromatic-C-ortho), 128.6 (s, broad, aromatic-C-meta), 126.9 (d, ¹J_{CP}=190.9 Hz), 65.4-63.7 (m, broad, backbone —CH₂—), 11.2 (d, J_{CP}=145.3 Hz,P—CH₃)

³¹P{H} NMR (CDCl₃, ppm): δ=32.4 (P—CH₃), 19.9 (P-Ph)

Representative NMR data of P(PhPPnₙ-grad-EtPPnₘ):

¹H NMR (CDCl₃, ppm): δ=7.9-7.6 (m, aromatic protons ortho), 7.6-7.3 (m, aromatic protons meta, para), 4.4-3.9 (m, backbone —CH₂—), 330-3.24 (s, broad, initiator —CH₃), 1.9-1.5 (in, P—CH₂—), 1.3-0.9 (m, P—CH₂—CH₃)

¹³C {H} NMR(CDCl₃, ppm): δ=132.8 (s, broad, aromatic-C-para), 131.8 (s, broad, aromatic-C-ortho), 128.9-128.2 (m, aromatic-C-meta), 126.9 (d, broad, ¹J_{CP} 191.2 Hz), 65.2-63.8 (m, backbone —CH₂—), 18.8 (d, ¹J_{CP}=142.9 Hz, P—CH₂—), 6.4 (s broad, P—CH₂—CH₃)

³¹P{H} NMR (CDCl₃, ppm): δ=35.2 (P-Et), 19.8 (P-Ph)

### Kinetic Measurements of Copolymerizations

To study the incorporation behavior of the different monomers during co-polymerization, a polymerization monomer mixture with the initiator prepared as mentioned above was transferred into a dry NMR tube under inert gas. This mixture was used to setup all NMR parameters (like shim and lock) at 263 K. The reaction was started by adding the calculated volume of catalyst solution (3 eq of DBU in respect to the initiator). The NMR tube was quickly placed in the NMR spectrometer and the experiments were started.

### Determination of Reactivity Ratios of Copolymerizations

The reactivity ratios were calculated by different nonterminal models following the instructions of, Jaacks,¹¹ Frey¹² or BSL¹³ and the Meyer-Lowry¹⁴ model as a terminal model. The protocol of Gleede et. al. was followed, and all methods used data from 0 up to 70% conversion to determine reactivity values.¹⁵ The average out of at least three models was used; the standard deviation was below 5%.

For P(PhPPn-EtPPn) r_{A} is 23.9±0.6 and r_{B} is 0.040±0.002

For P(PhPPn-MePPn) rp % is 4.48 ±0.02and r_{B} is 0.222±0.001

### T_{g} Measurements by DSC

PhPPn₃₀-grad-EtPPn₃₀: −20° C.
PhPPn₃₀-grad-MePPn₃₀: −10° C.
PhPPn₅₀-grad-MePPn₅₀: −10° C.

### Preparation of Micelles of Gradient Copolymers

Water-soluble gradient-co-polymers were dissolved in water at a desired concentration (usually 1-4 wt. %), mixed by vortex (Fisherbrand) and subsequently sonicated for 1 min in an ultrasonic bath (Branson) forming transparent, slightly opalescent dispersions.

Alternatively, polymers (1-4 wt. % of polymer related to final aqueous solution) were dissolved in 300 mg of acetone (VWR GPR recapture, 99%) and added to ultrapure water. The solution was sonicated for one minute and stirred overnight in an open vial to remove acetone. This procedure can be used for encapsulation of hydrophobic cargo, such as hydrophobic drugs. In this case, the hydrophobic cargo is dissolved in acetone together with polymer in required dose.

Data on Rₕ, PDI, T₁ and T₂ for the micelles of different gradient copolymers can be found in Table 1 and 2.

TABLE 1
Relaxation times of gradient-co-polymers.

| | | ³¹P con- | | | | | | |
|---|---|---|---|---|---|---|---|---|
| Phenyl- | Ethyl- | tent in | Rₕ | | T_{1, PH} | T_{2, PH} | T_{1, ET} | T_{2, ET} |
| units | units | polymer | [nm] | PDI | [s] | [s] | [s] | [s] |
| 30 | 30 | 20 wt. % | 9 | 0.04 | 0.9 | 0.09 | 1.8 | 0.9 |
| 44 | 56 | 20 wt. % | 9 | 0.07 | 0.9 | 0.17 | 1.8 | 0.8 |
| 60 | 40 | 19 wt. % | 11 | 0.02 | 1 | 0.06 | 1.8 | 0.8 |
| 30 | 70 | 21 wt. % | 11 | 0.2 | 0.9 | 0.23 | 2.1 | 1.3 |
| 32 | 28 | 20 wt. % | 8 | 0.04 | 0.9 | 0.13 | 1.7 | 0.8 |
| 29 | 31 | 20 wt. % | 8 | 0.06 | 0.9 | 0.14 | 1.8 | 1.0 |

TABLE 2
Relaxation times of gradient-co-polymers.

| | | ³¹P con- | | | | | | |
|---|---|---|---|---|---|---|---|---|
| Phenyl- | Methyl- | tent in | Rₕ | | T_{1, PH} | T_{2, PH} | T_{1, Me} | T_{2, Me} |
| units | units | polymer | [nm] | PDI | [s] | [s] | [s] | [s] |
| 30 | 30 | 22 wt. % | 11 | 0.26 | 0.9 | 0.29 | 2.2 | 1.2 |
| 47 | 53 | 22 wt. % | 9 | 0.22 | 0.9 | 0.27 | 2.2 | 1.2 |

The results of transverse relaxation T₂ in Table 1 and 2 are calculated based on monoexponential analysis of the measurement data.

FIG. 2 shows the result of a cell viability study with PhPPn₃₀-grad-EtPPn₃₀ micelles and monocytes, leukocytes and granulocytes. Viability at different micelle concentrations did not substantially differ from the control samples. Thus, no negative influence on cell viability was observed.

FIG. 3 depicts ¹H and ³¹P MRI of micelles of phenyl-co-ethyl phosphonate gradient co-polymer (30 phenyl units and 30 ethyl units, PhPPn₃₀-grad-EtPPn₃₀). Signals from both phenyl- and ethyl-phosphonate units can be distinguished based their chemical shift and added to a sum image forming the final MR image; better relaxation times from both monomers lead to a better signal.

Spatial localization of the imaging agents on an anatomical proton image is depicted in FIG. 4. For acquiring this image, a small amount of the micelles (around 4 mg) was injected in a physalis. As can be seen, the imaging agents can be localized on an anatomical proton image.

### Example 2: PEG-b-PPn Block Copolymer Micelles

Synthesis of Block-Copolymer poly(ethylene glycol-b-PhPPn) PEG-b-P(PhPPn):

Polymerization was performed according to a modified literature protocol.¹⁰ The PhPPn monomer were weighed in a flame-dried Schlenk-tube, dissolved in dry benzene and dried by lyophilization. The monomer was dissolved in dry dichloromethane to a total concentration of 4 mol L⁻¹. A stock solution of m-PEG₁₁₀ in dry dichloromethane was prepared with a concentration of 0.2 mol L⁻¹ and the calculated amount was added to the monomer solution. A stock solution of DBU in dry dichloromethane was prepared with a concentration of 0.2 mol L⁻¹. The monomer solution and the catalyst solution were set to the respective reaction temperature (in general 0° C.).

The polymerization was initiated by the addition of the calculated volume of catalyst solution containing 3.0 equivalents of DBU in respect to the initiator. Polymerization was terminated by the rapid addition of an excess of formic acid dissolved in dichloromethane with a concentration of 20 mg mL⁻¹. The colorless, amorphous polymers were purified by two times precipitation in cold diethyl ether and dried in vacuo. Yields ranged from 80% to 96%.

Representative NMR data of PEG₅₀₀₀-b-P(PhPPn):

¹H NMR (CDCl₃, ppm): δ=7.8-7.6 (m, aromatic protons ortho), 7.6-7.4 (m, broad, aromatic protons para), 7.4-7.2 (m, broad, aromatic protons meta), 4.3-4.9 (m, backbone —CH₂—), 3.6 (s, broad, PEG protons), 3.4 (s, initiator —CH₃)

³¹P{H} NMR (CDCl₃, ppm): δ=19.8

### Preparation of Micelles of Block Copolymers

Water-soluble gradient-co-polymers were dissolved in water at a desired concentration (usually 1-4 wt. %), mixed by vortex (Fisherbrand) and subsequently sonicated for 1 min in an ultrasonic bath (Branson) forming transparent, slightly opalescent dispersions.

Alternatively, polymers (1-4 wt. % of polymer related to the final aqueous dispersion) were dissolved in 300 mg of acetone (VWR GPR recapture, 99%) and added to ultrapure water. The solution was sonicated for one minute and stirred overnight in an open vial to remove acetone.

Data on Rₕ, PDI, T₁ and T₂ for the micelles of two different clock copolymers can be found in Table 3.

TABLE 2
Relaxation times of polyphenylphosphonate-b-polyethylene glycol

| PhPPn Repeat | ³¹P content in | | | | | |
|---|---|---|---|---|---|---|
| units | polymer | | Rₕ [nm] | PDI | T₁ [s] | T₂ [s] |
| 30 | 11 | wt. % | 10 | 0.05 | 1.2 | 0.05 |
| 50 | 9 | wt. % | 9 | 0.1 | 1.3 | 0.04 |

### Example 3: PEtPPn-b-PS Colloids

### PEtPPn-b-PS Synthesis¹⁹

A representative procedure for the synthesis of a PEtPn macro-CTA is described:

Ethyl ethylene phosphonate (1 g, 7.35 mmol, 60 eq) and 2-cyano-5-hydroxypental-2-yl dodecyl carbonotrithioate (48 mg, 0.123 mmol, 1 eq) were dissolved in anhydrous dichloromethane (1.83 mL) in an oven-dried 4 mL vial equipped with a magnetic stirring bar. The reaction mixture was homogenized by stirring at 20° C. followed by the addition of DBU (55 μL, 55.9 mg, 0.37 mmol, 3 eq) and the solution was stirred at room temperature for 1.5 h before the reaction mixture was quenched by the rapid addition of an excess of formic acid solution in dichloromethane (20 mg mL⁻¹). The crude product was purified by precipitation into cold diethyl ether (−20° C.) three times, and drying in vacuo to yield PEtPnC₆₂ macro-CTA as a yellow viscous liquid (0.95 g, 91%)
Representative NMR data of PEtPPn₆₂ macro-CTA:

¹H NMR (CDCl₃, ppm): δ=4.31-4.18 (m, backbone —CH₂—); 3.34 (t, J=7.4 Hz, initiator α-CH₂—S—), 1.88-1.78 (m, P—CH₂—), 1.24-1.15 (m, P—CH₂—CH₃).

³¹P{H} NMR (CDCl₃, ppm): δ=35.2

### Synthesis of PEtPPn₆₂-b-PS₃₄₅ by Aqueous Emulsion Polymerization

PEtPn₆₂ macro-CTA macroinitiator (129 mg, 0.01 mmol, 1 eq) and deionized water (2.73 g, 20 w/w %) were placed in a Schlenk flask and stirred until the macroinitiator was completely dissolved. A stock solution of VA-044 (10 mg mL-1) was prepared and VA-044 (1.57 mg, 0.002 mmol, 0.3 eq) was added to the reaction mixture. Styrene (0.53 g, 5.11 mmol, 350 eq) was weighed into a separate vial and added to the solution followed by stirring (1500 rpm) for 30 min. Then, the Schlenk flask was immersed in an ice bath and the solution was deoxygenized with nitrogen for 30 min and then immersed in an oil bath at 80° C. for 23h. Finally, the flask was placed in an ice bath and opened to air to terminate the polymerization.

¹H NMR (CDCl₃, ppm): δ=7.2-6.2 (m, aromatic protons); 4.4-4.1 (m, EtPPn backbone); 2.0-1.1 (m, PS backbone and EtPPn sidechain)

³¹P{H} NMR (CDCl₃, ppm): δ=35.2

Data on Rₕ, PDI, T₁ and T₂ for the micelles of the block copolymer can be found in Table 4.

TABLE 4
Relaxation times of PEtPPn-b-PS

| Et-PPn | ³¹P content in | | | | |
|---|---|---|---|---|---|
| Repeat units | polymer | Rₕ [nm] | PDI | T₁ [s] | T₂ [s] |
| 62 | 5 wt. % | 47 | 0.1 | 2.3 | 0.49 |

### Example 4: PhPPn-block-EtPPn

### PPn Block Copolymer Synthesis

Ring-Opening Polymerization Catalyzed with DBU

Polymerization was performed according to a modified literature protocol.¹⁰ The PhPPn and EtPPn monomer were weighed in two different flame-dried Schlenk-tubes, dissolved in dry benzene and dried by lyophilization. The monomers was dissolved in dry dichloromethane to a total concentration of 4 mol L⁻¹. A stock solution of initiator 2-methoxyethanole in dry dichloromethane was prepared with a concentration of 0.2 mol L⁻¹ and the calculated amount was added to the PhPPn monomer solution. A stock solution of DBU in dry dichloromethane was prepared with a concentration of 0.2 mol L⁻¹. The monomer solution and the catalyst solution were set to the respective reaction temperature (in general −10° C.). The polymerization was initiated by the addition of the calculated volume of catalyst solution containing 3.0 equivalents of DBU in respect to the initiator.

After 2.5 h the EtPPn solution was added rapidly to the reaction tube. Polymerization was terminated by the rapid addition of an excess of formic acid dissolved in dichloromethane with a concentration of 20 mg mL⁻¹. The colorless, amorphous polymers were purified by two times precipitation in cold diethyl ether and dried in vacuo. Yields ranged from 70% to 90%.

Representative NMR Data of P(PhPPnₙ-b-EtPPnₘ):

¹H NMR (CDCl₃, ppm): δ=7.9-7.6 (m, aromatic protons ortho), 7.6-7.3 (m, aromatic protons meta, para), 4.4-3.9 (m, backbone —CH₂—), 3.30-3.24 (s, broad, initiator —CH₃), 1.9-1.5 (m, P—CH₂—), 1.3-0.9 (m, P—CH₂—CH₃)

¹³C {H} NMR(CDCl₃, ppm): δ=132.8 (s, broad, aromatic-C-para), 131.8 (s, broad, aromatic-C-ortho), 128.9-128.2 (m, aromatic-C-meta), 126.9 (d, broad, ¹J_{CP}=191.2 Hz), 65.2-63.8 (m, backbone —CH₂—), 18.8 (d, ¹J_{CP}=142.9 Hz, P—CH₂—), 6.4 (s broad, P—CH₂—CH₃)

³¹P{H} NMR (CDCl₃, ppm): δ=35.2 (P-Et), 19.8 (P-Ph)

### Preparation of Micelles of Block Copolymers

Water-soluble gradient-co-polymers were dissolved in water at a desired concentration (usually 1-4 wt. %), mixed by vortex (Fisherbrand) and subsequently sonicated for 1 min in an ultrasonic bath (Branson) forming transparent, slightly opalescent dispersions.

Alternatively, polymers (1-4 wt. % of water used for dispersion) were dissolved in 300 mg of acetone (VWR GPR recapture, 99%) and added to ultrapure water. The solution was sonicated for one minute and stirred overnight in an open vial to remove acetone.

Data on Rₕ, PDI, T₁ and T₂ for the micelles of two different gradient copolymers can be found in Table 5.

TABLE 5
Relaxation times of block-co-polymers.

| | | ³¹P con- | | | | | | |
|---|---|---|---|---|---|---|---|---|
| Phenyl- | Methyl- | tent in | Rₕ | | T_{1, PH} | T_{2, PH} | T_{1, ET} | T_{2, ET} |
| units | units | polymer | [nm] | PDI | [s] | [s] | [s] | [s] |
| 50 | 50 | 20 wt. % | 11 | 0.05 | 1.1 | 0.05 | 1.9 | 1.0 |
| 20 | 80 | 21 wt. % | 30-70 | 0.3 | 1.1 | 0.17 | 2.2 | 1.5 |
| | | | nm | | | | | |

### LITERATURE

1. Senders, M. L.; Meerwaldt, A. E.; van Leent, M. M. T.; Sanchez-Gaytan, B. L.; van de Voort, J. C.; Toner, Y. C.; Maier, A.; Klein, E. D.; Sullivan, N. A. T.; Sofias, A. M.; Groenen, H.; Faries, C.; Oosterwijk, R. S.; van Leeuwen, E. M.; Fay, F.; Chepurko, E.; Reiner, T.; Duivenvoorden, R.; Zangi, L.; Dijkhuizen, R. M.; Hak, S.; Swirski, F. K.; Nahrendorf, M.; Perez-Medina, C.; Teunissen, A. J. P.; Fayad, Z. A.; Calcagno, C.; Strijkers, G. J.; Mulder, W. J. M., Probing myeloid cell dynamics in ischaemic heart disease by nanotracer hot-spot imaging. Nat. Nanotechnol. 2020, 15 (5), 398-405.
2. Flögel, U.; Temme, S.; Jacoby, C.; Oerther, T.; Keul, P.; Flocke, V.; Wang, X.; Bönner, F.; Nienhaus, F.; Peter, K.; Schrader, J.; Grandoch, M.; Kelm, M.; Levkau, B., Multi-targeted 1 H/19F MRI unmasks specific danger patterns for emerging cardiovascular disorders. Nat. Commun. 2021, 12 (1), 5847.
3. Higuchi, M.; Iwata, N.; Matsuba, Y.; Sato, K.; Sasamoto, K.; Saido, T. C., 19F and 1 H MRI detection of amyloid p plaques in vivo. Nat. Neurosci. 2005, 8 (4), 527-533.
4. Srinivas, M.; Morel, P. A.; Ernst, L. A.; Laidlaw, D. H.; Ahrens, E. T., Fluorine-19 MRI for visualization and quantification of cell migration in a diabetes model. Magn. Reson. Med. 2007, 58 (4), 725-734.
5. Koshkina, 0.; Lajoinie, G.; Bombelli, F. B.; Swider, E.; Cruz, L. J.; White, P.; Schweins, R.; Dolen, Y.; Dinther, E. v.; Riessen, N. K. v.; Rogers, S. E.; Fokkink, R.; Voets, I. K.; Eck, E. R. H. v.; Heerschap, A.; Versluis, M.; Korte, C. d.; Figdor, C.; Vries, I. J. M. d.; Srinivas, M., Multicore liquid perfluorocarbon-loaded multimodal nanoparticles for stable ultrasound and 19F MRI applied to in vivo cell tracking. Adv. Funct. Mater. 2019, 29 (19), 1806485.
6. Zhang, C.; Moonshi, S. S.; Wang, W.; Ta, H. T.; Han, Y.; Han, F. Y.; Peng, H.; Král, P.; Rolfe, B. E.; Gooding, J. J.; Gaus, K.; Whittaker, A. K., High F-Content Perfluoropolyether-Based Nanoparticles for Targeted Detection of Breast Cancer by 19F Magnetic Resonance and Optical Imaging. ACS Nano 2018, 12 (9), 9162-9176.
7. Ahrens, E. T.; Bulte, J. W. M., Tracking immune cells in vivo using magnetic resonance imaging. Nat. Rev. Immunol. 2013, 13 (10), 755-763.
8. Wolf, T.; Steinbach, T.; Wurm, F. R., A Library of Well-Defined and Water-Soluble Poly(alkyl phosphonate)s with Adjustable Hydrolysis. Macromolecules 2015, 48 (12), 3853-3863.
9. Steinbach, T.; Ritz, S.; Wurm, F. R., Water-Soluble Poly(phosphonate)s via Living Ring-Opening Polymerization. ACS Macro Letters 2014, 3 (3), 244-248.
10. Steinbach, T.; Ritz, S.; Wurm, F. R., Water-Soluble Poly(phosphonate)s via Living Ring-Opening Polymerization. Acs Macro Lett. 2014, 3 (3), 244-248.
11. Jaacks, V., A novel method of determination of reactivity ratios in binary and ternary copolymerizations. Die Makromolekulare Chemie: Macromolecular Chemistry and Physics 1972, 161 (1), 161-172.
12. Blankenburg, J.; Kersten, E.; Maciol, K.; Wagner, M.; Zarbakhsh, S.; Frey, H., The poly (propylene oxide-co-ethylene oxide) gradient is controlled by the polymerization method: determination of reactivity ratios by direct comparison of different copolymerization models. Polymer Chemistry 2019, 10 (22), 2863-2871.
13. Beckingham, B. S.; Sanoja, G. E.; Lynd, N. A., Simple and accurate determination of reactivity ratios using a nonterminal model of chain copolymerization. Macromolecules 2015, 48 (19), 6922-6930.
14. Meyer, V. E.; Lowry, G. G., Integral and differential binary copolymerization equations. Journal of Polymer Science Part A: General Papers 1965, 3 (8), 2843-2851.
15. Gleede, T.; Markwart, J. C.; Huber, N.; Rieger, E.; Wurm, F. R., Competitive Copolymerization: Access to Aziridine Copolymers with Adjustable Gradient Strengths. Macromolecules 2019, 52 (24), 9703-9714.
16. Ng, C.; Cousins, I. T.; DeWitt, J. C.; Glüge, J.; Goldenman, G.; Herzke, D.; Lohmann, R.; Miller, M.; Patton, S.; Scheringer, M.; Trier, X.; Wang, Z., Addressing Urgent Questions for PFAS in the 21st Century. Environ. Sci. Technol. 2021, 55 (19), 12755-12765.
17. Flögel U, Jacoby C, Gödecke A, Schrader J. In vivo 2D mapping of impaired murine cardiac energetics in NO-induced heart failure. Magn Reson Med. 2007; 57: 50-8.
18. Flögel U, Temme 5, Jacoby C, Oerther T, Keul P, Flocke V, Wang X, Bönner F, Nienhaus F, Peter K, Schrader J, Grandoch M, Kelm M, Levkau B. Multi-targeted 1 H/19F MRI unmasks specific danger patterns for emerging cardiovascular disorders. Nat Commun. 2021; 12: 5847.
19. Resendiz-Lara, D. A.; Wurm, F. R.Polyphosphonate-Based Macromolecular RAFT-CTA Enables the Synthesis of Well-Defined Block Copolymers Using Vinyl monomers; ACS Macro Lett. 2021, 10, 10, 1273-1279.

What is claimed is:

1. A method, comprising the step of:
using a P-containing polymer for measuring ³¹P-MRI, wherein the P-containing polymer is selected from polyphosphates, polyphosphonates, poly(phosphine oxide)s, polyphosphazenes, polyphosphinates, polyphosphoramidates, polyphosphorodiamidates, polyphosphoamides, polythionophosphates, and polythionophosphonates.

2. The method according to claim 1, wherein the amount of P in the polymer is at least 3 wt. %, preferably at least 5 wt. %, more preferably at least 10 wt. %, most preferably at least 15 wt. %.

3. The method according to claim 1, wherein the polymer has a T_{g} below 37° C., preferably below 35° C., more preferably below 30° C., most preferably below 25° C. or 20° C.

4. The method according to claim 1, wherein the polymer has a T₁ between 0.05 s and 5 s, a T₂ between 0.03 s and 3 s, and wherein T₁>T₂; preferably a T₁ between 0.1 s and 3 s, and a T₂ between 0.04 s and 1.5 s; and more preferably a T₁ between 0.5 s and 2.5 s, and a T₂ between 0.045 and 1.5, wherein T₁ and T₂ are the longitudinal and transverse relaxation times calculated using monoexponential decay.

5. The method according to claim 1, wherein the P-containing polymer is a polyphosphonate.

6. The method according to claim 5, wherein the polyphosphonate is a copolymer.

7. The method according to claim 6, wherein the polyphosphonate copolymer comprises at least two monomeric units A and B, wherein
monomeric unit 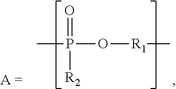
and
monomeric unit 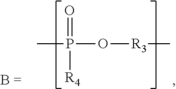
and wherein
R₁ and R₃ each independently are 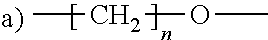
wherein n>1, preferably wherein 1<n<10; or 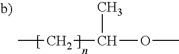
wherein n>0, preferably wherein 0<n<10; or 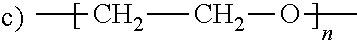
wherein n>0, preferably wherein 0<n<10; or 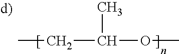
wherein n>0, preferably wherein 0<n<10;
and wherein
R₂ represents optionally substituted phenyl, optionally substituted benzyl, or optionally substituted phenethyl; and
R₄ represents straight or branched C₁₋₆ alkyl, straight or branched C₂₋₆ alkenyl, straight or branched C₁₋₆ alkoxy, or straight or branched C₁₋₆ alkanoyl.

8. The method according to claim 7, wherein
R₁ and R₃ are a) with n=2;
R₂ represents methylphenyl, dimethylphenyl, ethylphenyl, methylbenzyl, or phenethyl; and
R₄ represents straight or branched C1-4 alkyl, straight or branched C2-4 alkenyl, straight or branched C1-4 alkoxy, or straight or branched C1-4 alkanoyl, preferably wherein
R₂ represents phenyl, and
R₄ represents ethyl.

9. The method according to claim 7, wherein the polyphosphonate copolymer comprises from 30-70 mol % A, and from 30-70 mol % B, preferably from 35-65 mol % A, and from 35-65 mol % B, most preferably from 40-60 mol % A, and from 40-60 mol % B, the total of A and B adding up to 100 mol %.

10. The method according to claim 7, wherein the polyphosphonate copolymer is a diblock copolymer, a normal tapered block copolymer, or a gradient copolymer, preferably wherein the polyphosphonate copolymer is a gradient copolymer which has a gradient as defined in Gleede et al., Macromolecules 2019, 52 (24), 9703-9714 of block(-like), medium or hard, preferably of medium or hard.

11. The method according to claim 10, wherein the polyphosphonate copolymer is a gradient copolymer which has a value λ relative to the length of the minority monomeric unit as defined by Shull, Interfacial activity of gradient copolymers, Macromolecules 2002, 35, 8631-8639 of 0.5-1, preferably 0.6-1, more preferably of 0.8-1.

12. The method according to claim 7, wherein the polyphosphonate copolymer has a molecular weight (Mₙ) between 1000 and 100.000, and a dispersity between 1.01 and 10.00.

13. A polyphosphonate copolymer, which comprises:
two monomeric units A and B, wherein
monomeric unit 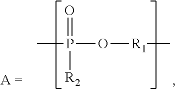
and
monomeric unit 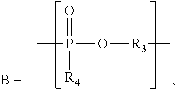
and wherein
R₁ and R₃ are 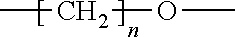
wherein n=2-4, and wherein
R₂ represents phenyl, benzyl, or phenethyl;
R₄ represents methyl, ethyl or propyl; and
wherein the copolymer comprises from 40-60 mol % A, and from 60-40 mol % B, and
wherein the polyphosphonate copolymer is a diblock copolymer, a normal tapered block copolymer, or a gradient copolymer, preferably wherein the polyphosphonate copolymer is a gradient copolymer; preferably wherein
R₁ and R₃ are 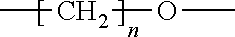
wherein n=2, and wherein
R₂ represents phenyl;
R₄ represents ethyl.

14. The polyphosphonate copolymer according to claim 13, wherein the polymer is a gradient copolymer, wherein r_{A} is between 4.3 and 24.4, and wherein r_{B} is between 0.03 and 0.25.

15. An aqueous suspension, comprising:
micelles of the polyphosphonate copolymer of claim 13, wherein the micelles have a hydrodynamic radius Rₕ as defined by ISO 22412:2008 of between 5 and 100 nm, a PDI of 0.001-0.5 as defined by ISO 22412:2008, and a concentration of between 1-500 mg/mL, preferably 5-490 mg/mL, more preferably 10-480 mg/mL.

16. The method according to claim 2, wherein the polymer has a T_{g} below 37° C., preferably below 35° C., more preferably below 30° C., most preferably below 25° C. or 20° C., wherein the polymer has a T₁ between 0.05 s and 5 s, a T₂ between 0.03 s and 3 s, and wherein T₁>T₂; preferably a T₁ between 0.1 s and 3 s, and a T₂ between 0.04 s and 1.5 s; and more preferably a T₁ between 0.5 s and 2.5 s, and a T₂ between 0.045 and 1.5, wherein T₁ and T₂ are the longitudinal and transverse relaxation times calculated using monoexponential decay, and wherein the P-containing polymer is a polyphosphonate.

17. The method according to claim 16, wherein the polyphosphonate is a copolymer, wherein the polyphosphonate copolymer comprises at least two monomeric units A and B, wherein
monomeric unit 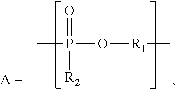
and
monomeric unit 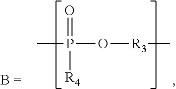
and wherein
R₁ and R₃ each independently are 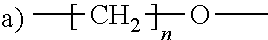
wherein n>1, preferably wherein 1<n<10; or 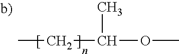
wherein n>0, preferably wherein 0<n<10; or 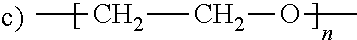
wherein n>0, preferably wherein 0<n<10; or 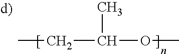
wherein n>0, preferably wherein 0<n<10;
and wherein
R₂ represents optionally substituted phenyl, optionally substituted benzyl, or optionally substituted phenethyl; and
R₄ represents straight or branched C₁₋₆ alkyl, straight or branched C₂₋₆ alkenyl, straight or branched C₁₋₆ alkoxy, or straight or branched C₁₋₆ alkanoyl.

18. The method according to claim 17, wherein the polyphosphonate copolymer comprises from 30-70 mol % A, and from 30-70 mol % B, preferably from 35-65 mol % A, and from 35-65 mol % B, most preferably from 40-60 mol % A, and from 40-60 mol % B, the total of A and B adding up to 100 mol % and wherein the polyphosphonate copolymer is a diblock copolymer, a normal tapered block copolymer, or a gradient copolymer, preferably wherein the polyphosphonate copolymer is a gradient copolymer which has a gradient as defined in Gleede et al., Macromolecules 2019, 52 (24), 9703-9714 of block(-like), medium or hard, preferably of medium or hard.

19. The method according to claim 18, wherein the polyphosphonate copolymer is a gradient copolymer which has a value λ relative to the length of the minority monomeric unit as defined by Shull, Interfacial activity of gradient copolymers, Macromolecules 2002, 35, 8631-8639 of 0.5-1, preferably 0.6-1, more preferably of 0.8-1 and wherein the polyphosphonate copolymer has a molecular weight (Mₙ) between 1000 and 100.000, and a dispersity D between 1.01 and 10.00.

20. An aqueous suspension, comprising:
micelles of the polyphosphonate copolymer of claim 14, wherein the micelles have a hydrodynamic radius Rₕ as defined by ISO 22412:2008 of between 5 and 100 nm, a PDI of 0.001-0.5 as defined by ISO 22412:2008, and a concentration of between 1-500 mg/mL, preferably 5-490 mg/mL, more preferably 10-480 mg/mL.
